(19) United States
(12) Patent Application Publication
(10) Pub. No.: US 2014/0135715 A1
(43) Pub. Date: May 15, 2014

(54) DEVICE FOR REDUCING RENAL SYMPATHETIC NERVE ACTIVITY

(71) Applicants: David Lambert, San Jose, CA (US); Fumiaki Ikeno, Menlo Park, CA (US)

(72) Inventors: David Lambert, San Jose, CA (US); Fumiaki Ikeno, Menlo Park, CA (US)

(21) Appl. No.: 14/232,282

(22) PCT Filed: Jul. 12, 2012
(86) PCT No.: PCT/US2012/046459

§ 371 Date: Jan. 12, 2014

Related U.S. Application Data
(60) Provisional application No. 61/507,104, filed on Jul. 12, 2011.

(51) Int. Cl. A61B 18/24; A61M 5/158

(52) U.S. Cl. CPC A61B 18/24; A61M 5/158
USPC 604/272; 606/15

(57) ABSTRACT

The present invention provides devices and methods for modulating or blocking renal sympathetic nerve activity by applying laser energy to the renal artery wall to effect ablation of the renal nerve or nerves. The devices and methods of the invention may be useful in the treatment of cardiovascular and renal disease resulting from hypertension. In addition the devices and methods of the invention may be useful for reducing reflex hypertension following cyclosporine administration after organ transplantation.

### FIELD OF THE INVENTION

The present invention relates generally to catheter-assisted devices and specifically to devices for modulating renal sympathetic nerve activity.

### BACKGROUND

Hypertension is now firmly linked with elevated renal sympathetic nerve activity in animal models and humans. While the underlying cause of this increased renal nerve activity is still unknown, the resulting increased blood pressure produces considerable morbidity including congestive heart failure (CHF) and kidney failure. Currently, the only approved method for treating hypertension is drug therapy, such as administration of diuretics, inhibitors of the rennin angiotensin system, and drugs that act on the heart to reduce cardiac workload. Although these drugs have proven beneficial, their use often results in up-regulation of reflex pathways that reduce their effectiveness over time.

Pharmacologically mediated hypertension has been observed in the clinic with the use of the anti-rejection drug cyclosporine (CsA) following organ transplantation. To be effective in preventing organ rejection, a patient must take the drug for the rest of his or her life. The utility of such treatment can be compromised when the patient develops a fulminate hypertensive response to cyclosporine therapy. It has recently been reported that in mouse models, CsA raises blood pressure by stimulating renal sensory nerve endings that contain synapsin-positive microvesicles. This increased afferent renal sympathetic nerve activity elicits a reflex afferent sympathetic vasoconstriction. In human patients, CsA administration following liver, kidney and heart transplants can cause an increase in blood pressure to pathological levels within days. Compounding the situation, the increased afferent sympathetic nerve activity causes renal artery constriction leading to increased renin release and a worsening of kidney function. Currently, drug therapy and diet modification are the only effective means to treat hypertension. Several classes of drugs exist to reduce high blood pressure. Diuretics have been a mainstay of therapy for many years and work by reducing blood volume by inhibiting sodium reabsorption in the kidney. Drugs that block the components of the renin-angiotensin system (RAS) are also commonly used. Renin inhibitors are a new class of drugs that act by blocking renin production by the kidney, while angiotensin converting enzyme inhibitors (ACE) inhibitors act by blocking conversion of angiotensin I to the active form angiotensin II. Angiotensin receptor antagonists block the vasoconstriction produced by angiotensin peptides binding to receptors in the vasculature. Drugs that act on the heart to decrease workload are used in hypertension treatment as well as later stages of congestive heart failure.

In experimental animals with hypertension, renal denervation has been shown to provide an immediate benefit in lowering blood pressure by blocking the increased sympathetic nerve activity to and from the kidneys. This approach has not been used in human patients, however, due in part to the risks involved with denervation surgery. Moreover, severed human renal nerves are able to regenerate, but often do so in an abnormal or pathological way. Not only does this decrease the potential efficacy of surgical denervation, but may also lead to pain and other serious side effects. Therefore, renal denervation is not considered a valid treatment for uncontrolled hypertension in human patients.

Devices previously described for ablating a renal nerve have used radiofrequency energy. Operation of such devices requires insertion into a renal artery of a human patient and rotation such that the electrode makes contact with the luminal surface of the renal artery. Radiofrequency energy is thus applied to the renal artery wall, heating the arterial wall, thereby damaging the underlying renal nerve. The electrode is then repositioned in the renal artery, and radiofrequency energy applied to another point on the wall of the renal artery. However, the inability to direct the device to the precise position of the artery where the renal nerve lies makes it necessary to make repeated burns to ensure that the renal nerve is ablated. Radiofrequency energy is thus applied in a pattern of discrete burns circumferentially around the lumen of the renal artery, resulting in damage to the renal nerve, but also resulting in significant damage unrelated to the therapeutic renal ablation effect.

A significant limitation of existing devices is the inability to visualize the renal nerve. Radiofrequency energy is delivered to the renal artery wall in a series of burns, with the intent of delivering energy through the renal artery wall to the renal nerve lying on the surface of the renal artery, thus damaging the nerve and preventing nerve conduction. However, this method is limited by the skill of the operator without the aid of visual feedback, in making a sufficient number of burns to ensure that the nerve is damaged and in effect relying on chance. Additionally, unnecessary damage to the wall of the renal artery occurs when energy is applied to areas of the renal artery wall not underlying the renal nerve.

### Imaging

The ability to visualize the renal nerves of a patient in order to treat elevated sympathetic renal nerve activity associated with refractory hypertension would be of great benefit in devices, systems and methods for ablating renal nerve activity. Such an improvement would bring the device within an acceptable level of risk of damage to unrelated structures in the patient relative to therapeutic benefit. High frequency ultrasound (HFU) and optical coherence tomography (OCT) are two exemplary methods that have been used to visualize structures within human tissue. High-frequency ultrasound relies on reflected sound waves to visualize subsurface structures in tissue, whereas OCT relies on reflected light energy. Both techniques rely of the differences in reflected energy from different tissue types to visualize tissue, organs and other structures in a patient. In general, tissues of different composition reflect and scatter light differently and these differences can be detected and processed to create an image in either two or three dimensions (2-D or 3-D) for identifying and locating the imaged tissue.

An advantage of OCT is that it has the ability to produce images of much higher resolution (better than 10 μm) than other imaging methods such as MRI or ultrasound. To date, OCT has been used in a variety of biomedical applications and many variations have been described. For example, time-domain OCT has been used to visualize the structures in the anterior and posterior areas of the eye, as well as structures beneath the skin and in the gastrointestinal tract. In frequency domain OCT broadband interference is acquired with spectrally separated detectors that can improve imaging speed dramatically. Recent developments in OCT technology have led to the development of Fourier-domain OCT, which allows information from the full depth of a scan to be acquired within a single exposure. Time encoded frequency domain OCT tries to combine the advantages of standard time domain and Fourier-domain by encoding the spectral components in time rather than by spatial separation.

Devices, systems and methods for controllably and selectively reducing or blocking renal nerve activity while minimizing side effects due to damage to non-target tissue by precisely and accurately targeting renal nerves would offer an improvement in the treatment of hypertension over currently available technology.

### SUMMARY OF THE INVENTION

The present invention provides a probe for ablating a renal nerve in a patient, comprising a generally hollow body formed from a biologically compatible material, having a proximal end, a distal end and a length therebetween, the body comprising at least one lumen; said distal end having disposed therein an ablation means; said proximal end having disposed therein a means for transmitting information to and/or receiving information from said ablation means. The probe generally has a size of 4 Fr to 10 Fr. In certain embodiments, the probe also includes an aperture, such as a window, disposed through the body through which the ablation means can be projected.

The probe body is typically flexible and optionally is bent on the distal end to allow placement against the renal artery wall.

Typically, the ablation means is directed outwardly through the aperture, and is optionally retractable. In certain aspects of the invention, the ablation means can be retractably advanced outwardly in a radial direction from within the body of the probe through the aperture, which can place the ablation means in contact with the inner wall of a vein or artery, or in contact with a renal nerve or nerves.

The means for transmitting information to and receiving information from the ablation means can include a digital interface, a power supply, a fluid infusion means for infusing fluids through the at least one lumen, or a combination thereof. The information received by the proximal end can include information about the location of the renal nerve, and can permit visualization of a treatment endpoint. The information can be imaging information such OCT imaging information or ultrasound imaging information.

In certain aspects of the invention, the probe also includes an imaging means disposed at the distal end, which can include optical coherence tomography (OCT). Such OCT can detect the birefringence boundary between two different tissues, such as between nerve tissue and muscle or between nerve tissue and connective tissue.

The ablating means can be electrically or thermally conductive and/or it can deliver at least one form of ablation energy such as laser light, ultrasound, microwave energy or electrical current, wherein the ablation means is adapted for cutting through or coagulating biological material. The laser light can be produced by a solid state laser, free electron laser, diode laser, chemical laser, gas laser, or metal vapor laser. The laser typically operates at least one wavelength from about 700 nm to 2500 nm, and in certain aspects of the invention will operate over a range or band selected from the group consisting of a wavelength band of 880 nm to 935 nm, a wavelength band of 1150 nm to 1230 nm, a wavelength band of 1690 nm to 1780 nm, and a wavelength band of 2250 nm to 2450 nm. In certain aspects, laser energy heats the renal nerve or nerve tissue to a temperature of at least 50° C. but not more than 90° C.

In certain aspects of the invention the ablation means selectively targets lipid rich tissue, such as nerve tissue or myelin.

In certain embodiments the ablation means can be activated by transmitting a fluid through a lumen of the ablation probe. In certain aspects, the ablation means includes a retractable ablation element, such as an electrode adapted for delivering electrical energy to the renal nerve; an electrode adapted for delivering thermal energy to the renal nerve; or a needle adapted for delivering fluid to the renal nerve. For example, the needle can be hollow and deliver a drug to the renal nerve or the fluid can be a heated fluid, a cooled fluid or a neurotoxic fluid. In various embodiments the ablation element is an electrode that delivers thermal energy, electrical energy or a combination of energies to the renal nerve.

Optionally, the probe also includes a flexible, expandable temperature sensing element disposed on the distal end. The probe can also include a lumen adapted for accepting a positioning guide wire for positioning the distal end of the probe at a treatment site in the patient.

The probe will generally be adapted for insertion into an artery or vein of the patient and advanced to a treatment site in the renal artery. In operation, the probe is advanced to the treatment site through an introducer catheter.

The probe can include one or more fiber optic cables in communication with the means for transmitting and receiving information. In certain embodiments the fiber optic cable(s) transmits and delivers imaging information and/or ablation energy.

The probe can include one or more reversibly inflatable balloons disposed on the distal end of the probe, which is in fluid communication with the at least one lumen. The balloon may be adapted for positioning and immobilizing the probe at the treatment site, such as by disposing the balloon opposite the ablation means. In other embodiments, the balloon is adapted for occluding blood flow. Typically, the balloon is inflated by means of a fluid such as saline or a gas such as CO₂.

The probe can also include a lumen in fluid communication with an irrigation means disposed at the proximal end for irrigating the treatment site with a fluid such as saline, and at least one irrigation port adjacent to the aperture.

The probe can also include a radioopaque marker that can be visualized within the body of the patient.

### BRIEF DESCRIPTION OF THE DRAWINGS

FIG. 1A is an illustration of one embodiment of an ablation probe according to the present invention. FIG. 1B shows the distal end of an ablation probe having a bias. FIG. 1C shows the distal end of an ablation probe that includes a wire to direct placement against the wall of the renal artery.

FIGS. 2A-2C illustrate an embodiment of an ablation probe employing two fiber-optic cables. FIG. 2A shows a cross section of the ablation probe. FIG. 2B show the top of the distal end of the probe. FIG. 2C shows the bottom of the probe.

FIGS. 3A-3C illustrate an embodiment of an ablation probe employing a single fiber optic cable to bidirectionally carry both imaging signal and laser energy. FIG. 3A shows a cross section of the ablation probe. FIG. 3B show the top of the distal end of the probe. FIG. 3C shows the bottom of the probe

FIG. 4 is an illustration of the proximal end of an ablation probe according to an embodiment of present invention.

FIG. 5 is a schematic of an OCT system adapted for use in visualizing the renal nerves of a patient.

FIG. 6 is an illustration of a balloon cuff for use with the current invention.

FIG. 7 is an illustration of a device according to the invention deployed in a renal artery of a patient.

FIG. 8 is an image taken from a human subject in the lateral prone position, showing the renal artery, renal vein and kidneys.

FIG. 9 is an image of the renal artery shown with distances from the lumen wall to the renal nerves.

FIG. 10 shows H&E staining of pig renal artery from the same section that OCT imaging was captured, showing orientation of main renal nerve branch in relation to renal artery lumen.

FIG. 11 shows OCT imaging of ex vivo pig renal artery. Freshly excited pig renal arteries with kidneys attached were imaged using an OCT imaging catheter. The catheter was inserted through the aorta and advanced into the renal artery.

FIG. 12 shows histological (H&E) staining of human renal artery, showing a cross section of the artery and the renal nerves.

FIG. 13 shows OCT imaging of ex vivo human renal artery, showing renal nerves and a cross section of the renal artery. Separation of the renal nerves from the renal artery is an artifact of fixation in formalin.

### DETAILED DESCRIPTION

The present invention provides devices for modulating renal sympathetic nerve activity, which can be used to reduce blood pressure and associated diseases, such as cardiovascular disease and kidney disease. In certain embodiments, a device according to the present invention reduces renal sympathetic nerve activity. In other embodiments, renal sympathetic nerve activity is completely blocked. The present invention also provides devices for ablation of the renal nerve or nerves of patients, for example, to effect reduction of elevated renal sympathetic nerve activity associated with hypertension and heart failure.

As used herein, “renal sympathetic nerve activity” refers to the transmission of nerve impulses through the renal nerve. The terms “modulate”, “modulation of”, modulating” and the like, in the context of renal sympathetic nerve activity, refer to increasing or decreasing the transmission of nerve impulses through the renal nerve, and in particular, partially or completely blocking the transmission of nerve impulses through the renal nerve. Partially blocking the transmission of nerve impulses through the renal nerve includes non-ablative injury or damage to the renal nerve. In certain embodiments, the renal nerve is modulated by severing the renal nerve physically (e.g., by cutting), electrically (e.g. by applying an electrical to the nerve), optically (e.g., by applying light to the nerve), thermally (e.g. by applying heat or cold to the nerve), or chemically (e.g., by exposing the nerve to a chemical or pharmacological agent such as a neurotoxin).

In another embodiment of the invention, an ablation probe is provided for ablating a renal nerve and/or nerves. The ablation probe is a generally hollow body, (e.g. a shaft or catheter), having one or more lumens and having at a first (proximal) end adapted for connection to a power supply with a digital interface disposed thereon; and, a second (distal) end terminating distally with a tip, which includes an ablation means, which can be formed from an electrically or thermally conductive material. Optionally, the second end of the ablation probe may include a port for local drug infusion and the first end may include a drug infusion means. The ablation probe can be fitted with an imaging means, such as optical coherence tomography (OCT), to enable imaging and precise placement of the device near the renal nerve or nerves.

The present invention provides an ablation probe for ablating the renal nerve or nerves of a patient. Such probes are useful for reducing elevated renal sympathetic nerve activity associated with hypertension and heart failure. Ablation of the renal nerve may be accomplished through directed energy, such as laser energy, or alternatively through high frequency electrical current from an electrical source. In certain embodiments, ablation is effected by transmission of a fluid through the ablation probe of the invention to activate the ablation device. Alternatively, ablation may be effected by application of high frequency ultrasound. The ablation probe is positioned in the body using an imaging means, such as optical coherence tomography (OCT), to enable precise positioning of the ablating device near the renal nerve or nerves of a patient.

### Ablation of Renal Nerve

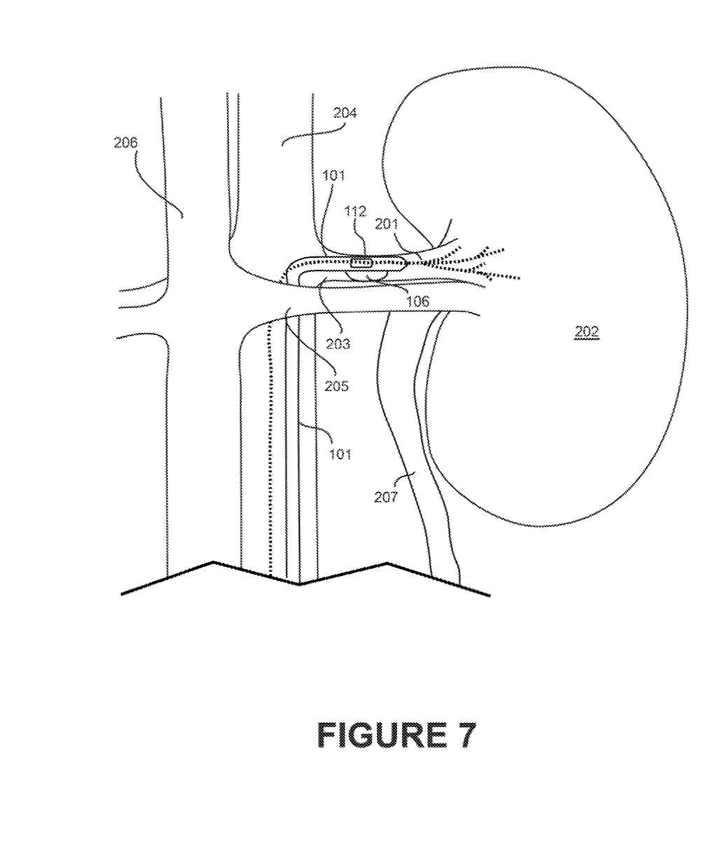

Referring to FIG. 7, the ablation probe 101 is shown deployed in the renal artery of a patient. The ablation probe includes an ablating means 112 for ablation of the renal nerve(s) 201. For anatomical reference, FIG. 7 also shows the kidney 202, which is supplied with blood via the renal artery 203, that branches from the abdominal aorta 204. Filtered blood is returned to the circulation via the renal vein 205, which connects to the vena cava 206. Waste thereby travels to the bladder via the ureter 207.

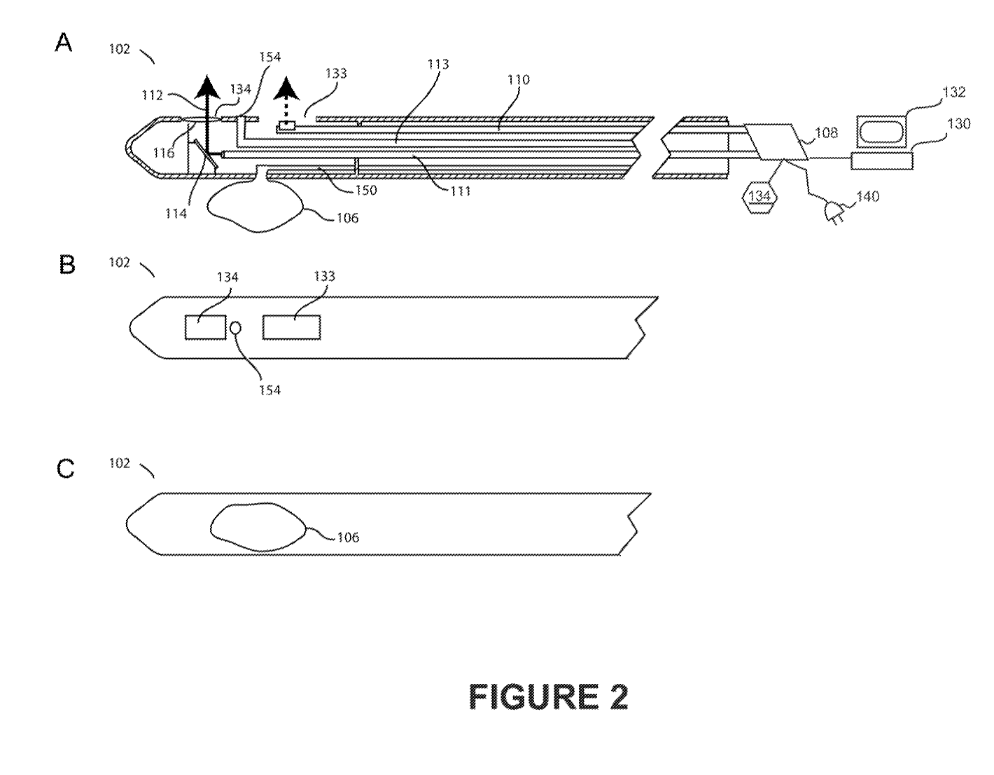

According to certain embodiments of the present invention, the ablation probe 101 is adapted for percutaneous insertion into an artery of a subject, (e.g. a human patient) followed by advancement to a treatment site, which is typically a position within a renal artery. Ideally, the ablation probe is size that allows for insertion into the renal artery of a patient, e.g. 5 Fr to 10 Fr. The ablating means 112 is positioned at the treatment sites (see FIG. 7, such as a position within the renal artery near the hilum or ostium of the artery where the artery connects to the abdominal aorta. Inflation of a balloon 106 acts to oppose the ablating means against the lumen wall of the renal artery. Activation of a laser source contained within or adjacent to the device causes laser energy to be directed through a fiber optic cable contained within the ablation probe 101 and focused at the distal end 102, wherein it exits through a lens 116 (see FIG. 2A) onto the wall of the renal artery. Transmission of laser energy through the arterial wall is absorbed in the renal nerve or nerves, damaging the nerve and preventing transmission of nerve signals. In this embodiment, an imaging window 133 on the distal end 102 allows imaging of the renal nerve or nerves through the renal artery wall, for example by OCT, as a means of directing laser energy, or an alternative energy such as high frequency ultrasound or thermal energy, into the renal nerve or nerves.

In use, the balloon 106 is inflated to a pressure that opposes the distal end 102 of the ablation catheter against the renal artery wall, but is still of low enough pressure to allow the catheter to be rotated within the renal artery. Energy, such as laser energy, is delivered to the walls of the blood vessel at the treatment site, and thereby delivers energy to the blood vessel (e.g. the renal artery wall). Energy applied to the blood vessel wall is conducted therethrough, thereby delivering the energy to the nerves that the lie on the surface of the blood vessel. For example, when energy is delivered to the renal artery wall, such energy is conducted to renal nerves laying on the outer surface of the renal artery. When sufficient energy is delivered to the nerve, coagulation of the nerve results, or alternatively ablation results, thereby reducing or completely blocking transmission through the nerves. Previous inventions have described methods of delivering laser energy to tissue that is preferentially absorbed in tissue containing a high water content, such as fat. Of particular usefulness to the present invention is the use of laser energy that is preferentially absorbed in fatty tissue, or tissue containing a high lipid content, such as the myelin layer surrounding the renal nerve or nerves, or the renal nerve itself, as a means to coagulate the renal nerve tissue, while sparing tissue low in lipid content, such as the renal artery wall or adventitia.

Radiofrequency energy has previously been applied to the lumen of the renal artery as a means to heat the renal nerves, thus ablating overactive renal nerve activity associated with hypertension. There are several drawbacks to this method, the major one being that RF energy delivered via an electrode to the renal artery wall had to be of sufficient frequency and power to diffuse through the renal artery wall to reach the renal nerves lying on the adventitial layer. This may result in considerable artery wall tissue necrosis. Second, RF energy, when absorbed into biological tissue such as the renal artery wall, will disperse throughout the artery wall, potentially causing intimal and medial damage over a wide area of the renal artery wall. Another drawback is the inability to visualize the renal nerve or nerves. When energy, such as RF energy, is applied in a pattern of several discrete burns around the circumference of the renal artery wall, in a manner designed to maximize the potential of damaging the renal nerves without heating the entire 360° circumference of the renal artery wall simultaneously, the likelihood of producing a considerable hypertrophic response and renal artery stenosis is increased.

As used herein, “renal nerve” refers to an individual nerve or group of nerves or nerve branches that transmit nerve signals to and/or from the kidneys. The kidney is innervated by the renal plexus, which is intimately associated with the renal artery. As is well known in the art, preganglionic neuronal cell bodies are located in the intermediolateral cell column of the spinal cord. Preganglionic axons pass through the paravertebral ganglia to become the lesser thoracic splanchnic nerve, least thoracic splanchnic nerve, first lumbar splanchnic nerve and second lumbar splanchnic nerve and travel to the aortocorenal ganglion. Postganglionic neuronal cell bodies are located in the aorticorenal ganglion. Postganglionic axons enter the renal plexus and are distributed to the renal vasculature.

In certain embodiments of the device, the distal end 102 of the ablation probe is fitted with a balloon 106 or balloons, over which the ablating means 112 is disposed. FIG. 7. According to these embodiments, the body of the ablation probe 101 contains one or more lumens in communication distally with the balloon or balloons, and proximally with a fluid infusion port or other balloon inflation means. Infusion of a liquid through the lumen and into the balloon, fills the balloon causing it to expand. Inflation of the balloon 106 forces the ablating means 112 of the ablation probe 101 against the blood vessel wall for application of energy to the treatment site. Deflation of the balloon allows the ablating means to be repositioned within the renal artery.

Optionally, the ablation probe can be fitted with an imaging means, such as optical coherence tomography (OCT) or intravascular ultrasound (IVUS), to enable imaging and precise placement of the device within the renal artery. When present, the imaging means is in communication with a digital interface and image processing means through a fiber optic cable.

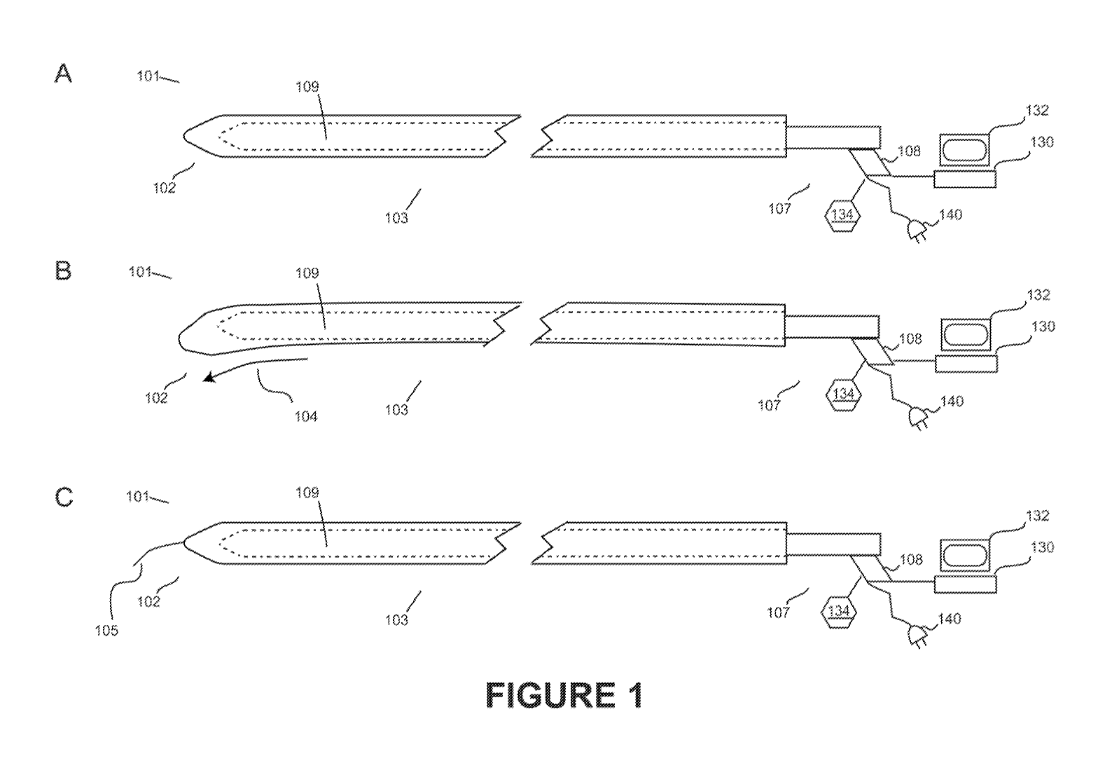

In yet another embodiment of the device, the body of the ablation probe, and in particular the distal end 102, has a bias or bend built into the distal end to allow placement of the distal end against a wall of the renal artery. See FIG. 1. The bias may be built into the plastic material of the catheter. In this embodiment, the ablation probe is inserted into an artery, such as the renal artery, through an introducer catheter containing a lumen. Over the distal end of this introducer catheter a balloon can be attached, such that the balloon surrounds the catheter. In use, the introducer catheter is inserted into the body and advanced to the renal artery, whereby the balloon of the introducer catheter is then inflated to form a seal in a region of the renal artery proximal to the treatment region. Inflation of the balloon forms a seal and blocks blood flow through the renal artery. The ablation probe is then inserted through the introducer catheter and the distal end 102 is advanced to a region distal to the inflated balloon of the introducer catheter. The operator then positions the distal end 102 of the ablation probe against the renal artery wall to allow for visualization of the renal nerve or nerves, and for delivery of energy to the renal nerve or nerves.

In certain embodiments of the invention, the device includes a means for cooling the renal artery wall before, during or after treatment. Pre-cooling enables the operator to maintain the treatment site at a reduced temperature such that subsequent heating with an ablation means energy source, (e.g. a laser), does not result in thermal damage to the renal artery wall. Cooling of the treatment site during and after application of ablation energy is important as well, as it prevents and/or reduces conduction of heat delivered to the deeper tissues of the renal artery wall from heating the more superficial layers of the renal artery wall and causing damage to the endothelial layer.

Cooling of the treatment zone, whether through pre-cooling, during treatment, or post-cooling, may be accomplished through various cooling methods that will be know in to the skilled artisan. In one embodiment, a cold liquid, such as saline, is used to irrigate the treatment site.

Referring to FIGS. 2A and 2B, irrigation port(s) 154 disposed through the body of the ablation probe 101, adjacent to the ablation window 134, and in communication with a fluid lumen of the catheter 113, allow for cooling of the treatment area either before, during, or after treatment. Cold liquid, such as saline, typically in the temperature range of 0° C. to 5° C., is injected into a lumen 113 of the ablation probe 101 which is in communication with irrigation port(s) 154. In use, flushing of the treatment site, such as the luminal wall of the renal artery, with cooled saline can occur before, during, or after treatment of the renal nerves.

Referring to FIG. 1A, one embodiment of a device according to the present invention is shown from a side view. In particular, ablation probe 101 is generally a hollow body 103 (i.e. a catheter) having a first (proximal) end 102, a second (distal) end 107, and at least one lumen 109 within the body 103. The ablation probe can be made from any biologically inert material, such as a medical grade plastic or other polymer. The catheter itself is flexible, and optionally has a bias or bend 104 built into the distal end 102 (arrow) (FIG. 1B). The bend 104 built into the distal end 102 of the catheter is designed directs the placement of the distal end of the ablation probe against the wall of the renal artery. In certain aspects of the invention, a wire 105 is disposed on the distal end of the probe to direct placement against the wall of the renal artery (FIG. 1C).

The proximal end 107 of the ablation probe includes a digital interface 108 in communication with a power source 140. Optionally, the digital interface is in communication with a computer 130 including a programmable logic controller or microprocessor and/or a monitor 132, e.g. for temperature display and control, energy control, and/or processing and visualizing images from an imaging means. In another embodiment, the digital interface 108 is also in communication with an energy source 134, such as a laser.

The distal end 102 of the ablation probe 101 includes an ablating means 112 for ablating a nerve, such as a renal nerve (FIG. 2A). In certain aspects of the invention, the ablating means 112 is located within the body 103 of the ablating probe and projects outwardly toward an arterial wall through an ablation window 134, which window traverses through the body 103 of the ablation probe. In certain embodiments of the invention, the ablating means 112 is a laser that is directed through the ablation window 134 by a mirror 114 and/or lens 116 as illustrated in FIG. 2A. In other embodiments, the ablating means comprises a needle, a heated and/or cooled element, an electrode or a combination thereof. Optionally, in these other embodiments, the ablating means can be projected through the ablating window (e.g. mechanically) to contact the arterial wall or through the arterial wall to contact a renal nerve.

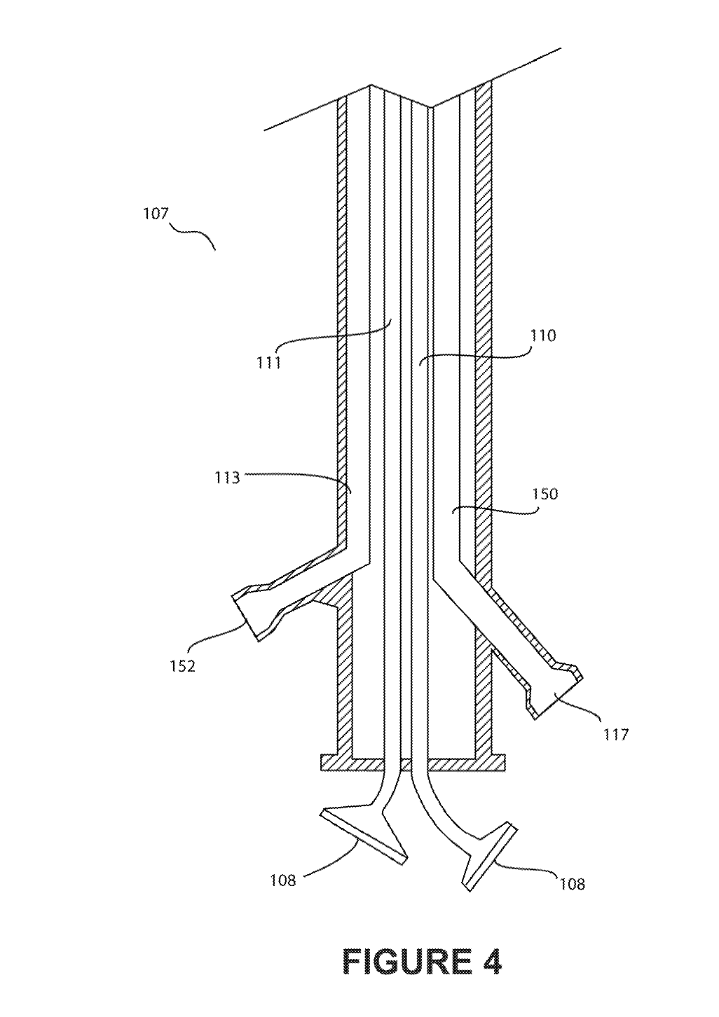

In the embodiment illustrated in FIGS. 2A and 2C, a positioning balloon 106 is also disposed on the distal end 102 of the ablation probe. When inflated, positioning balloon 106 opposes an imaging window 133 formed through the body of the ablating probe and the ablation window 134 such that the windows contact the wall of the renal artery. The positioning balloon 106 is in fluid communication with a positioning balloon lumen 150 located within the body of the ablation probe. The positioning balloon lumen 150 is in fluid communication on the proximal end 107 of the probe with an inflation injection port 117, to allow for inflation of the balloon 106 (FIG. 4).

In certain embodiments illustrated in FIG. 2A, imaging fiber optic cable 110 and ablation fiber optic cable 111 are disposed within the body of 101 and in communication on the proximal end with at least one digital interface 108. Imaging fiber optic cable 110 carries imaging signal, for example OCT imaging signal, from the digital interface 108 in communication with a computer 130 at the proximal end 107 of the ablation probe. The imaging fiber optic cable terminates at the imaging window 133 on the distal end 102 of the ablation probe. Fiber optic cable 111 carries ablation energy, such as laser light, and is in communication on the proximal end with a digital interface 108 and computer 130. A mirror, 114, disposed at the distal end of the ablation probe is positioned to reflect the laser energy through the ablation window 134 outwardly toward the wall of the renal artery. A lens 116 can be present in the path of the laser to filter the laser light to deliver laser energy capable of photocoagulating tissue. Imaging window 133 and ablation window 134 are placed as close together as possible so that ablation energy is delivered to the renal artery wall in a position as close as possible to the area visualized by the imaging means, such as OCT. Typically the distance between the imaging window 133 and the ablation window 134 will be between 1 mm and 10 mm; in certain aspects the ablation window imaging window 133 and the ablation window 134 are between 1 mm and 5 mm apart, and most frequently 1 mm to 2 mm apart.

### Ablation Means

Ablation of the renal nerve may be accomplished through directed energy, such as high frequency electrical current from an electrical source. In other embodiments, ablation of the renal nerve may be accomplished through directed application of laser energy or high frequency ultrasound energy.

In certain embodiments, modulation of renal nerve transmission is accomplished using thermal heating mechanisms, which may include raising the temperature of renal nerve fibers above a desired threshold, for example, above a body temperature of about 37° C. e.g., to achieve non-ablative thermal injury, or above a temperature of about 45° C., such as above about 50° C., above about 55° C., about 60° C., above about 65° C. or a higher temperature to achieve ablative thermal injury.

In other embodiments, modulation of renal nerve transmission is accomplished using thermal cooling mechanisms, which may include non-freezing thermal slowing of nerve transmission and/or non-freezing thermal nerve injury, as well as freezing thermal nerve injury. Thermal cooling mechanisms may include reducing the temperature of target renal nerve fibers below a desired threshold, for example, below the body temperature of about 37° C., such as between 1° C. and about 36° C., typically between about 1° C. and about 20° C. Thermal cooling mechanisms may also include reducing the temperature of the target neural fibers below about 0° C., e.g., to achieve freezing thermal injury or ablation. The skilled artisan will recognize that degree of non-ablative injury or ablative injury to the renal nerve can be controlled by the duration of exposure to thermal heating and/or cooling. In certain embodiments, thermal heating and/or cooling is applied continuously. Alternatively, thermal heating and/or cooling can be applied intermittently, with periods of treatment alternated with periods of non-treatment or recovery. The invention contemplates that intermittent application of thermal heating and or cooling will reduce damage to surrounding non-target tissue. The invention also contemplates that thermal modulation can be accomplished using alternating cycles of heating and cooling of the renal nerve target. In such embodiments, the probe may include parallel means for heating the renal nerve and cooling the renal nerve, or a single ablation means that is suitable for both heating and cooling the renal nerve, such as a thermally conductive needle that is in communication with both a heating source and a cooling source, or a controllable thermal source that can apply both heating and cooling.

In certain embodiments, ablation is facilitated by transmission of a fluid through an ablation probe of the invention to activate the ablating means, such as through inflation of a balloon or infusion/injection of a fluid. In such embodiments, inflation of the balloon is accomplished by means of a heated fluid, such as saline, to allow for ablation of the renal nerve or nerves. Ablation may also be accomplished through use of a cooled fluid, such as saline.

In other embodiments, ablation is facilitated by means of a laser. For the purposes of this invention, the energy used for renal nerve ablation may also be chosen from microwave, infrared, visible or ultraviolet energy, generally in the range of 100 nm to 100 micrometer wavelength. Laser types suitable for use in the ablation probes of the invention include solid state lasers, chemical lasers, gas lasers, diode lasers, or metal vapor lasers. In particular, the laser may be a solid state laser consisting of, for example, a yttrium aluminum garnet (YAG) host combined with an element such as erbium (Er) or Thulium (Tm), producing a Er:YAG or Tm:YAG laser. The wavelength of the energy output shall be selected from, but not limited to, a range of 700 nm to 2500 nm, inclusive. Wavelengths of laser light contemplated for use in the devices of the invention include bands between 880 nm to 935 nm, 1150 nm to 1230 nm, 1690 nm to 1780 nm, or 2250 nm to 2450 nm, which are wavelengths preferentially absorbed by lipid cells.

In still other embodiments of the invention, the ablating means 112 delivers high frequency ultrasound or radiofrequency energy to the renal nerve. In certain embodiments of the invention, the ablating means 112 has disposed therein at least one electrode in electrical communication with the digital interface. In the embodiment illustrated in FIG. 1C, a flexible metal wire is optionally disposed and, when present, extends distally therefrom. The wire 105, when present provides a means of navigation of the catheter into a blood vessel.

### Imaging Means

Optical coherence tomography (OCT) is an optical signal acquisition and processing method allowing the generation of extremely high-quality, micrometer-resolution, three-dimensional images from within optical scattering media, such as biological tissue. In practice, an optical beam is directed at the tissue, and a small portion of the light that reflects from sub-surface features is collected. The glare of scattered light causes optically scattering materials, such as biological material, to appear opaque or translucent. Only the reflected (non-scattered) light is coherent and is digitally processed to obtain an image of subsurface features. OCT is able to penetrate and obtain subsurface imaging at least 1 to 2 mm below the surface in biological tissue.

In biological and biomedical imaging applications, OCT allows for imaging on the micrometer level in translucent and semi-translucent tissues. The tissue penetration capability of OCT is based on low-coherence interferometry, in which light from a broadband source is split between illuminating the tissue of interest and a reference path. The interference pattern of light reflected back from the sample and light from the reference delay contains information about the location and scattering amplitude of the light scatterers in the sample. In conventional (time-domain) OCT, this information is extracted by scanning the reference path delay and detecting the resulting interferogram pattern as a function of that delay.

In another embodiment, the ability to visualize the renal nerve may be enhanced by the use of polarization-sensitive OCT. The use of an OCT system that enhances the birefriengence boundary between the renal nerve and or nerves and surrounding tissue is particularly useful for enhancing visualization of tissues of differing composition, such as the myelin sheath surrounding a renal nerve versus surrounding connective tissue of vascular smooth muscle.

OCT is particularly effective in imaging the renal nerve or nerves when the light source is able to penetrate as far as possible through the renal artery wall. In certain embodiments, the light source penetrates at least 0.5 mm; in other embodiments penetration of at least 1 mm is obtained; in yet other embodiments, the light source penetrates to a depth of 2, 3 or 4 mm.

In one aspect of the invention off-axis OCT imaging is used and the fiber optic cable carrying the imaging light source is positioned off-center from the catheter body to place it as near to the wall of a catheter as possible. Such placement allows the OCT imaging light source to approach the wall of the renal artery as closely as possible and thereby allows deeper penetration of the OCT imaging signal into the tissue. In certain embodiments the OCT imaging light source directly contact luminal wall of the renal artery. Greater resolution of structures located beyond the luminal wall of the renal artery is obtained compared to other OCT imaging systems in which the OCT fiber optic cable is further from the arterial tissue.

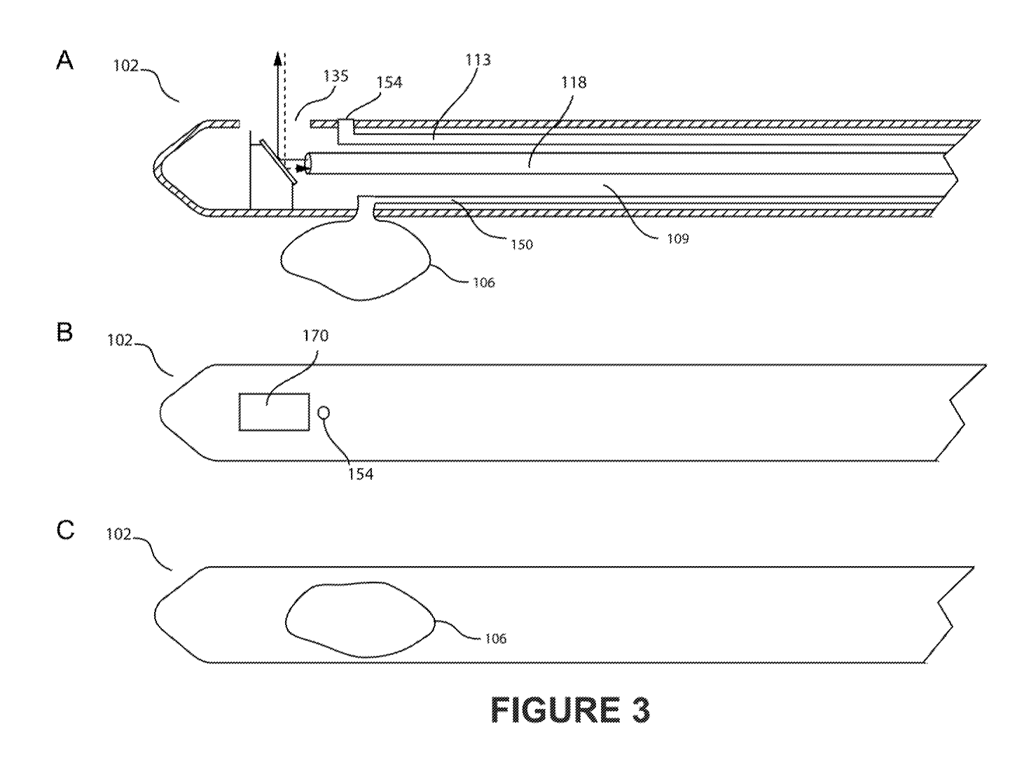

In another embodiment of the device, the imaging signals and ablation energy are carried in a single fiber optic cable. As illustrated in FIG. 3A, a bidirectional, bifunctional fiber optic cable 118 is provided to carry an imaging signal, for example OCT signal, as well as an ablation energy, such as laser energy, through a single window 155. In use, controls at the proximal end of the probe (e.g. the computer), are in communication with the ablation means 112 and imaging means 115 through digital interface 108, which allows the operator to switch between imaging mode and treatment mode.

Referring to FIG. 4, the proximal end 107 of the ablation probe 101 according to the present invention is illustrated. Specifically, fluid injection port 117 is disposed at the distal end of the probe in fluid communication with a balloon inflation lumen 150 which traverses the length of the probe, and is in fluid communication with a positioning balloon 106 disposed at the proximal end 107 of probe 101. Inflation of balloon 106 is accomplished, e.g. by connecting a fluid-filled (e.g. saline-filled) syringe to inflation port 117 and injecting fluid into the positioning balloon 106. In use, an optional pressure gauge (not shown) may be used to control the pressure within the fluid injection port 117 and positioning balloon 106. The positioning balloon 106 is typically inflated to 0-10 psi. Also shown in FIG. 4 is saline injection port 152, which is in fluid communication with irrigation lumen 113, to allow injection of cold saline into the treatment site. Irrigation of treatment site is accomplished injecting cold saline into injection port 152 which is in fluid communication with irrigation lumen 113. Irrigation lumen 113 delivers cold saline to one or more irrigation ports at the distal end 102 of the ablation probe 101 adjacent to the ablation window 134 (FIGS. 2A and 2B) or ablation/imaging window 135 (FIGS. 3A and 3B). Also shown in FIG. 2A are fiber optic cables 110 and 111, which carry OCT imaging signal and laser energy, respectively. Fiber optic cables 110 and 111 terminate at a digital interface or interfaces 108, which may be connected to a computer 130.

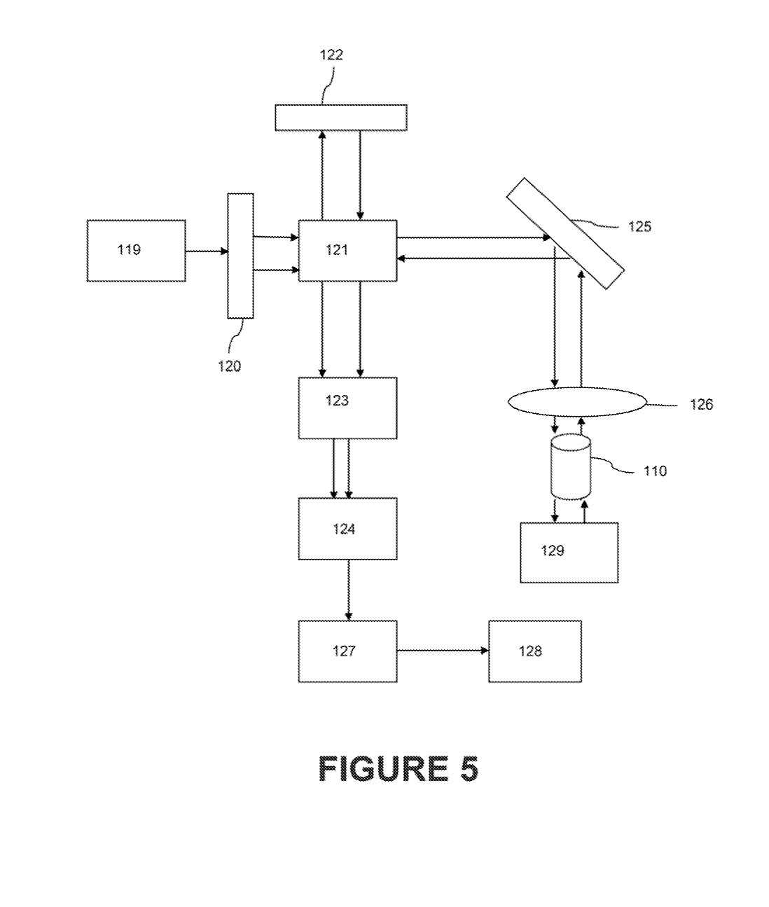

FIG. 5 shows a generalized schematic of an OCT system adapted for visualizing the renal nerves of a patient is shown. In particular, light source 119 emits a light beam through a collimation lens 120, after which the light beam passes through a beam splitter 121 and is split into two separate light beams, a reference beam and a sample beam. The reference beam is reflected off of reference mirror 122 and passes through a beam reducer 123 to a photo detector 124. Sample beam is reflected off of scanning mirror 125 and passes through a lens 126. Sample beam then passes through fiber optic cable 110 of the ablation probe, through which it is focused on the renal artery wall. Reflected light from the target tissue 129, for example structures located within or proximal to the renal artery wall such as a renal nerve or nerves, passes through lens 126 and is reflected off scanning mirror 125 to the beam splitter 121, after which it passes through beam reducer 123 and photo detector 124. Both reference beam and reflected light from sample tissue pass to signal processor 127, where the signals are analyzed and an image of the target tissue is displayed on computer display 128.

A limitation of using OCT in cardiovascular applications is the scattering or diffraction of light passing through blood, in particular turbid blood flow that may be encountered in an artery of a patient. In certain embodiments of the invention, a balloon cuff, consisting of a catheter having one or more lumens passing through the body of the catheter, is deployed in the renal artery of a patient before treatment.

### Balloon Cuff

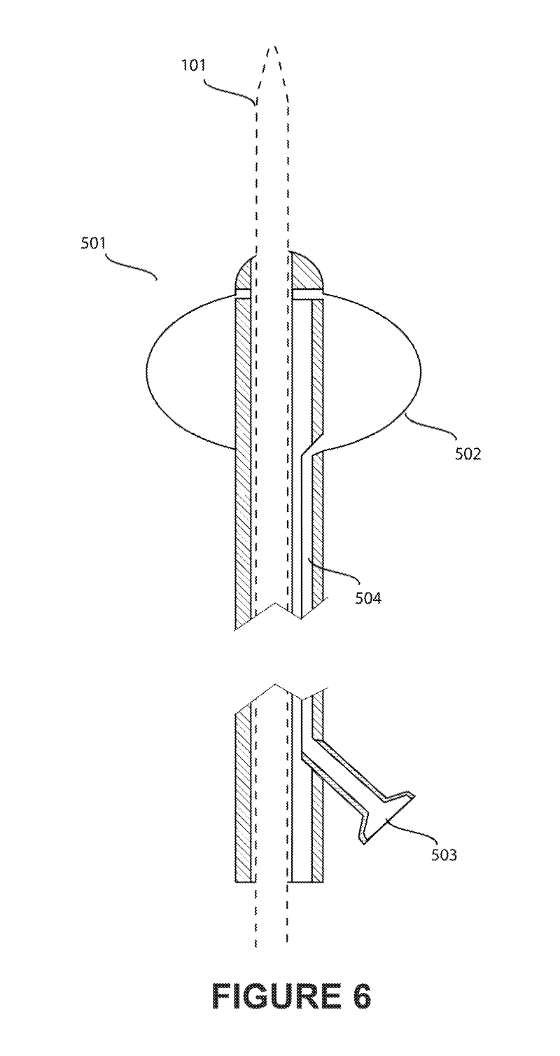

FIG. 6 shows an embodiment of a balloon cuff for use with the ablation probe of the present invention device. In general, balloon cuff 501 is a hollow body catheter made of a suitable inert material such as medical grade plastic, in the size range from 5 Fr to 11 Fr to allow insertion percutaneously and advancement to the renal artery of a patient. Balloon cuff includes an inflatable, donut shaped balloon 502, surrounding the distal end of the balloon cuff and in communication with a lumen 504 to allow for inflation of the balloon through infusion port 503. Balloon 502 is typically inflated with a fluid, such as saline. Alternatively, balloon 502 may be inflated with a gas, such as CO₂ or ambient air. In use, balloon inflation cuff is inserted into the proximal portion of the renal artery and the balloon cuff inflated to block blood flow into the renal artery. The ablation probe 101 (shown with dotted lines) is then passed through a lumen of the balloon cuff to a point distal to the inflated cuff. In this manner, blood flow through the renal artery is disrupted for a period of time, e.g., lasting no more than 10 minutes, to reduce or block the interference of circulating blood on the OCT light source. Inflation of the balloon cuff occurs through use of a liquid, such as saline, or by a gas, such as CO₂. In practice, after the ablation probe 101 is inserted in the renal artery of a patient, the balloon cuff is inflated to a sufficient pressure to block blood flow through the renal artery as a means of reducing the interference of blood flow on the OCT light source.

### EXAMPLES

### Example 1

### Sonogram of Human Renal Vasculature to Investigate Anatomy and Best Approach to Renal Artery

Increased renal sympathetic nerve activity is a major contributor to essential hypertension. However, the underlying cause of this centrally mediated increase in sympathetic nerve activity is unknown. The kidneys are highly sensitive to changes in blood pressure, and afferent sympathetic nerve traffic leading from the kidneys to the brain regulates reflex efferent sympathetic arterial constriction and vascular tone. The renal nerve branches from the renal plexus lying near the aorta and travels along the renal artery to the kidney. The renal nerve lies in close proximity to the renal artery and branches into a network, or plexus, of nerves nearer the kidney.

The main branch of the renal nerve lies immediately on top of the advential layer of the renal artery, encased in a layer of fat. Previous studies focused on determining whether a retroperitoneal approach, combined with imaging technology, could be used to precisely locate the renal nerves within a human patient for the purposes of applying directed energy to the nerve to block sympathetic nerve signals transduction along the renal nerve. Preclinical studies were conducted using intravascular ultrasound (IVUS) and sonography to image the renal nerve.

Materials and Methods

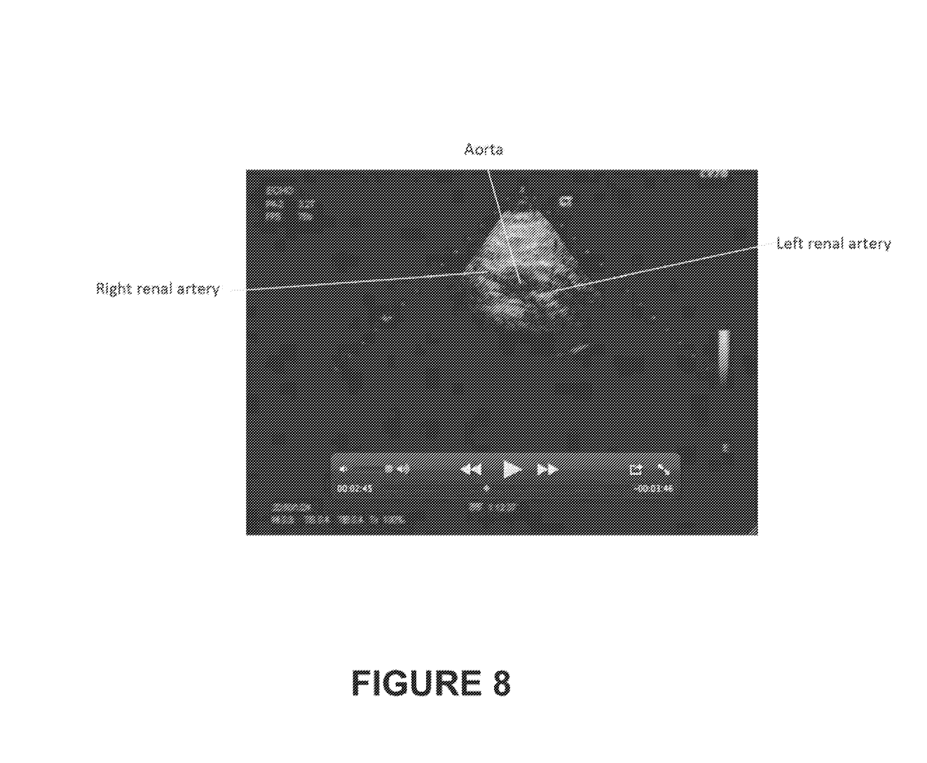

Human kidneys from adult males were visualized in the supine and lateral supine position using an Accuson CV70 Sonogram System. FIG. 8 is an image taken from a human subject in the lateral prone position, showing the renal artery, renal vein and kidneys. Arterial blood shows as red and veinous blood as blue. These studies indicated that the position of the kidneys relative to the renal artery makes a retroperitoneal approach to the renal nerve difficult. The adult renal artery has a width of approximately 6-7 mm. The length of the renal artery from the hilum of the kidney to the aorta is approximately 5 cm for the right renal artery and 4 cm for the left renal artery.

### Example 2

### Histology of Pig Renal Artery Showing Renal Nerves

Materials and Methods.

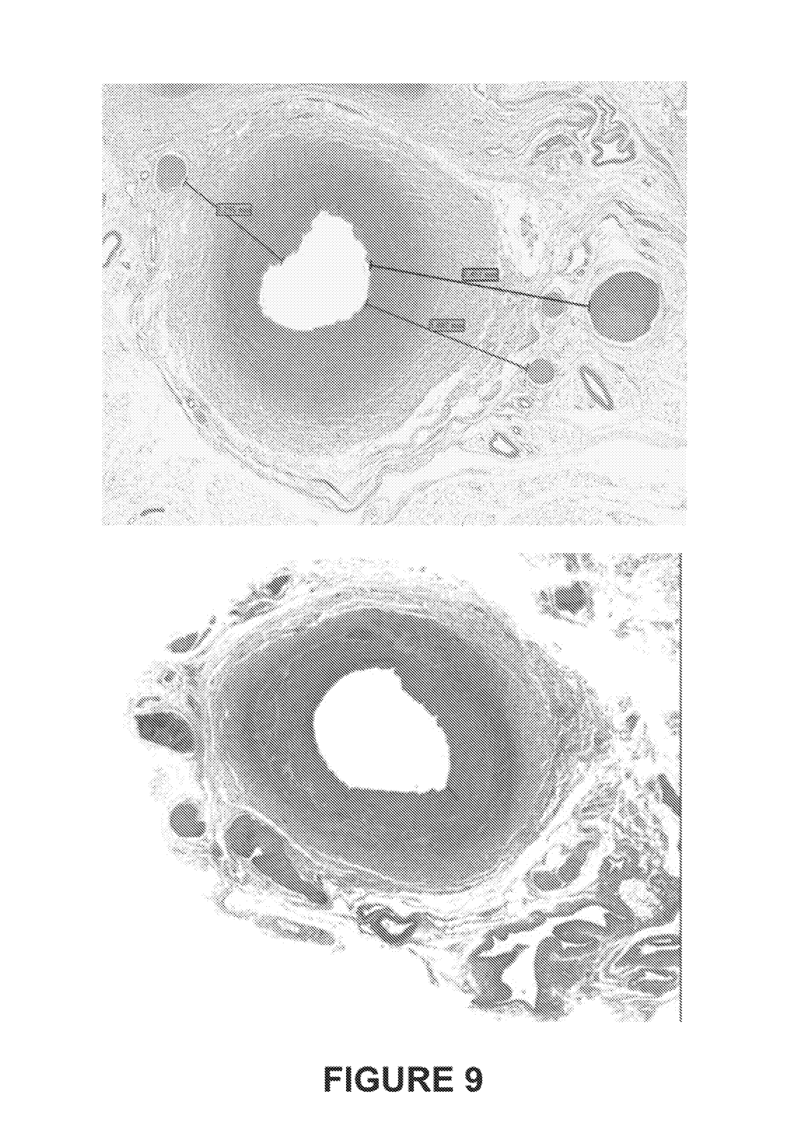

The renal arteries were excised from Gottingen minipigs and placed in formalin for histological (H&E) staining. In FIG. 9, the renal artery is shown with distances from the lumen wall to the renal nerves.

### Optical Coherence Tomography of Renal Nerves

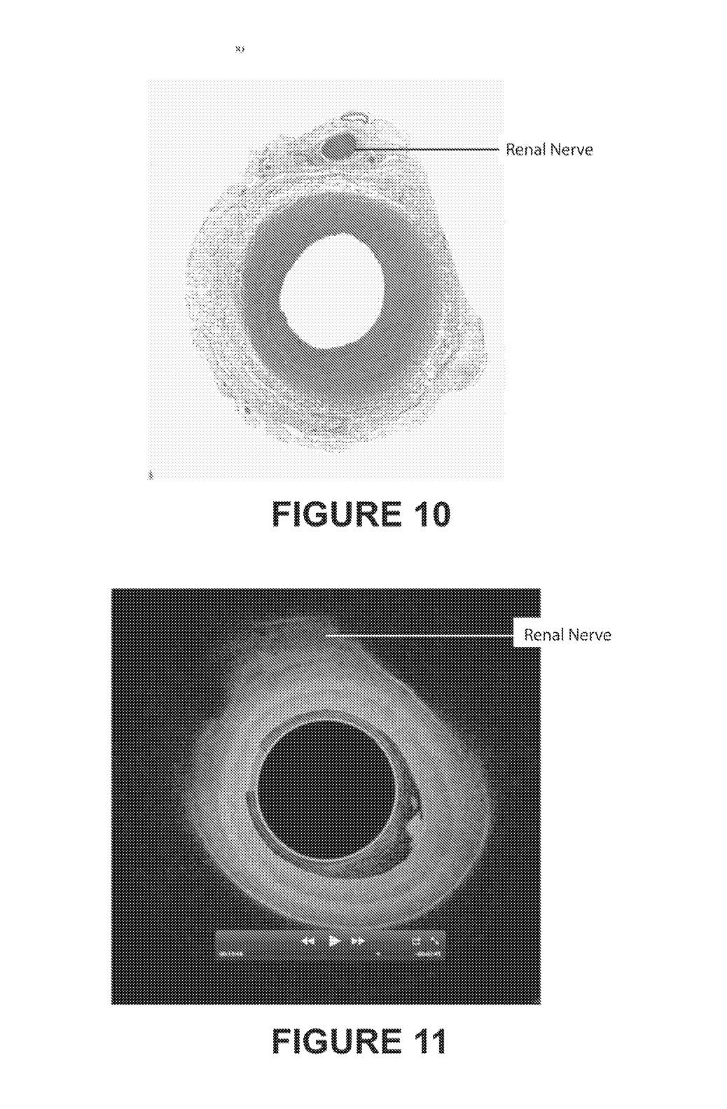

To investigate the ability to visualize the renal nerve through the renal artery wall, freshly harvested pig renal arteries were obtained, still attached to the aorta and kidneys. The samples were placed on a dissection tray and excess fat was dissected from the renal artery. An incision was made in the abdominal aorta to facilitate placement of an OCT imaging catheter inside the renal artery. FIG. 11 is OCT imaging of the renal nerve, showing the location of the renal nerve in relation to the artery lumen. FIG. 10 shows H&E staining of the same section of renal artery verifying the ability to image the renal nerve through the renal artery wall.

### Example 3

### Histology of Human Renal Artery Showing Renal Nerves

Materials and Methods.

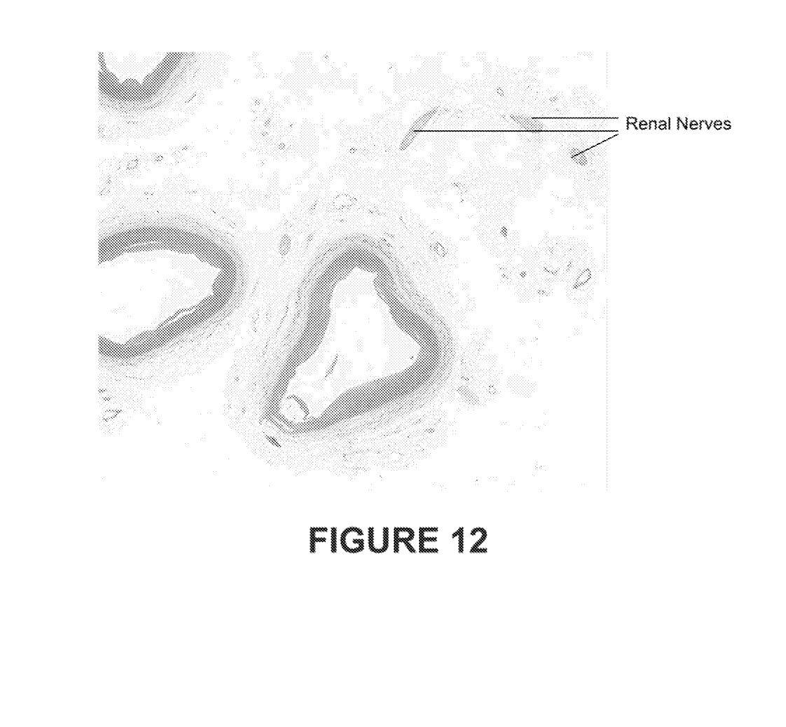

The right renal artery from an 80 year old male cadaver is shown after histological (H&E) staining. Of significance to the usefulness of this device is the almost complete lack of intimal thicking or hyperplasia, FIG. 12 shows H&E staining of a section of the right renal artery. Renal nerves are shown. Separation of renal nerves from the renal artery is an artifact of tissue shrinkage and separation during formalin fixation.

### Optical Coherence Tomography of Renal Nerves

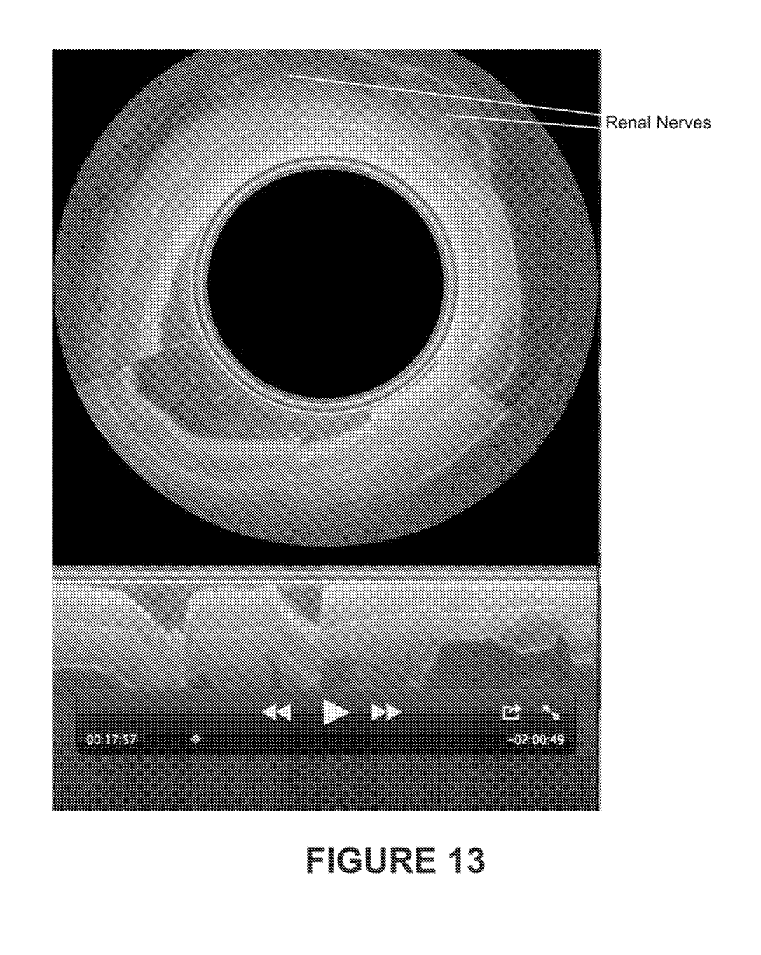

To investigate the ability to visualize the renal nerve through the renal artery wall, the renal artery with renal nerves attached from an 80 year old male cadaver was thawed in room temperature saline and placed on a tray for optical coherence visualization. FIG. 13 is OCT imaging of the renal nerves, showing the location of the renal nerves in relation to the artery lumen.

### REFERENCES

U.S. Pat. No. 7,717,948 Methods and Apparatus for Thermally-Induced Renal Neuromodulation May/2010.
U.S. Pat. No. 7,697,145 System for Fourier Domain Optical Coherence Tomography April/2010.
U.S. Pat. No. 7,801,590 Optical Coherence Tomographic Detection of Cells and Killing of the Same September/2010.
2004/0034341 Method and Apparatus for the Selective Targeting of Lipid-Rich Tissues February/2004.
De Boer, Johannes F., et al., “Two-dimensional birefriengence imaging in biological tissue by polarization-sensitive optical coherence tomography,” Jun. 15, 1997, Optics Letters, vol. 22, No. 12, pp. 934-936.
Everett, M. J. et al., “Birefringence characterization of biological tissue by use of optical coherence tomography,” 1998, Optics Letters, vol. 23 Issue 3, pp. 228-230.

1. A probe for ablating a renal nerve in a patient, comprising:
a generally hollow body formed from a biologically compatible material, having a proximal end, a distal end and a length therebetween,
the body comprising at least one lumen;
said distal end having disposed therein an ablation means;
said proximal end having disposed therein a means for transmitting information to and/or receiving information from said ablation means.

2. The probe of claim 1, further comprising an aperture disposed through the body through which the ablation means can be projected, wherein the ablation means is optionally retractable or comprises a retractable element, and is optionally projected outwardly in a radial direction from within the body through the aperture and optionally the ablation means contacts the wall or a vein, the wall of an artery or the renal nerve when projected outwardly from within the body.

3. (canceled)

4. (canceled)

5. (canceled)

6. (canceled)

7. (canceled)

8. The probe of claim 1, wherein the means for transmitting information to and/or receiving information from said ablation means comprises at least one of a digital interface, a power supply, and a fluid infusion means for infusing fluids through the at least one lumen and the information is optionally the location of the renal nerve.

9. The probe of claim 1, wherein the ablating means is electrically or thermally conductive.

10. (canceled)

11. The probe of claim 10, wherein the probe further comprises an imaging means disposed at the distal end, wherein the imaging means optionally comprises optical coherence tomography (OCT), wherein the OCT optionally detects the birefringence boundary between two different tissues, which two different tissues are optionally nerve tissue and muscle or nerve tissue and connective tissue.

12. (canceled)

13. (canceled)

14. (canceled)

15. The probe of claim 10, wherein the information received by the proximal end permits visualization of a treatment endpoint, wherein the information is optionally imaging information selected from the group consisting of OCT information and ultrasound information.

16. (canceled)

17. The probe of claim 1, wherein of the ablation means delivers at least one form of ablation energy selected from the group consisting of: laser light, ultrasound, microwave energy and electrical current, wherein the ablation means is adapted for cutting through or coagulating biological material, wherein optionally the ablation means selectively targets lipid rich tissue, which lipid rich tissue is optionally nerve tissue or myelin.

18. (canceled)

19. (canceled)

20. The probe of claim 17, wherein the laser is a solid state laser, chemical laser, gas laser, free electron laser, diode laser, or metal vapor laser, and optionally the laser operates at at least one wavelength from about 700 nm to 2500 nm.

21. (canceled)

22. The probe of claim 20, wherein laser operates at wavelength band selected from the group consisting of a wavelength band of 880 nm to 935 nm, a wavelength band of 1150 nm to 1230 nm, a wavelength band of 1690 nm to 1780 nm, and a wavelength band of 2250 nm to 2450 nm

23. The probe of claim 17, wherein the laser energy heats the nerve tissue to a temperature of at least 50° C. but not more than 90° C., wherein optionally the nerve tissue is the renal nerve.

24. The probe of claim 1, wherein the ablation means is activated by transmitting a fluid through the at least one lumen.

25. The probe of claim 1, further comprising:
a flexible, expandable temperature sensing element disposed on the distal end;
at least one reversibly inflatable balloon disposed on the distal end of the probe optionally dispose opposite the ablation means, wherein the balloon is in fluid communication with the at least one lumen, wherein the balloon is optionally adapted for positioning and immobilizing the robe at the treatment site and optionally occluding blood flow; or
at least one radioopaque marker that can be visualized within the body of the patient.

26. (canceled)

27. The probe of claim 1, wherein the probe is adapted for insertion into an artery or vein of the patient and advanced to a treatment site in the renal artery and is optionally advanced to the treatment through an introducer catheter.

28. (canceled)

29. The probe of claim 1, wherein the at least one lumen is adapted for accepting a positioning guide wire for positioning the distal end of the probe at a treatment site in the patient.

30. The probe of claim 1, wherein probe comprises at least one fiber optic cable in communication with the means for transmitting and/or receiving information, which is optionally at least one of imaging information and ablation energy.

31. (canceled)

32. The probe of claim 1, further comprising at least one reversibly inflatable balloon disposed on the distal end of the probe optionally disposed opposite the ablation means, wherein the balloon is in fluid communication with the at least one lumen, wherein the balloon is optionally adapted for positioning and immobilizing the probe at the treatment site and optionally occluding blood flow, wherein the balloon is optionally inflated by means of a fluid or a gas, and is optionally selected from saline and CO₂.

33. (canceled)

34. (canceled)

35. (canceled)

36. (canceled)

37. (canceled)

38. The probe of claim 1, wherein the at least one lumen is in fluid communication with an irrigation means disposed at the proximal end for irrigating the treatment site with an irrigation fluid and at least one irrigation port adjacent to the aperture, wherein the irrigation fluid is optionally saline.

39. (canceled)

40. (canceled)

41. (canceled)

42. (canceled)

43. The probe of claim 1, wherein the body of the probe is flexible or is bent on the distal end to allow placement against the renal artery wall.

44. (canceled)

45. (canceled)

46. The probe of claim 2, wherein the ablation means is selected from the group consisting of: an electrode adapted for delivering electrical energy to the renal nerve; an electrode adapted for delivering thermal energy to the renal nerve; and a needle adapted for delivering fluid to the renal nerve, wherein the needle is optionally hollow and the fluid is optionally selected from the group consisting of a drug-containing fluid, a neurotoxic fluid, a heated fluid, and a cooled fluid.

47. (canceled)

48. (canceled)

49. (canceled)

50. A ablation system, comprising an ablation probe according to claim 1 and an inflatable balloon catheter for stopping blood flow.
